(19) United States
(12) Patent Application Publication
(10) Pub. No.: US 2023/0056999 A1
(43) Pub. Date: Feb. 23, 2023

(54) METHOD AND DEVICE FOR PROCESSING RAMAN DATA OF EOSINOPHILS BASED ON ARTIFICIAL INTELLIGENCE

(71) Applicant: Apolon Inc., Seoul (KR)

(72) Inventors: Jun Ki KIM, Seoul (KR); Miyeon JUE, Seoul (KR)

(73) Assignee: Apolon Inc., Seoul (KR)

(21) Appl. No.: 17/820,461

(22) Filed: Aug. 17, 2022

(30) Foreign Application Priority Data
Aug. 18, 2021 (KR) 10-2021-0108750
Sep. 14, 2021 (KR) 10-2021-0122266

(51) Int. Cl. G16H 50/20; G16B 40/00; G16B 5/20

(52) U.S. Cl. CPC G16H 50/20; G16B 40/00; G16B 5/20

(57) ABSTRACT

Disclosed is a method for processing Raman data of eosinophil based on artificial intelligence, the method being executed by a device, the method including generating Raman data by performing Raman analysis using a specific wavelength on eosinophils isolated from blood of a diagnosed person, pre-processing the generated Raman data, assigning a weight to the pre-processed Raman data for each of components including a nucleus, a cell membrane, a granule, and a background, classifying data for each component based on a result of assigning the weight, extracting data in which the component is the granule based on a classified result; and determining whether a specific disease has occurred in the diagnosed person through eosinophil characteristics of the diagnosed person based on the extracted data.

### CROSS-REFERENCE TO RELATED APPLICATIONS

A claim for priority under 35 U.S.C. § 119 is made to Korean Patent Application Nos. 10-2021-0108750 filed on Aug. 18, 2021 and 10-2021-0122266 filed on Sep. 14, 2021 in the Korean Intellectual Property Office, the entire contents of which are hereby incorporated by reference.

### BACKGROUND

Embodiments of the inventive concept described herein relate to a method and apparatus for processing Raman data of eosinophils based on artificial intelligence.

In general, asthma is treated to be aimed at relieving symptoms and controlling inflammation, with steroids, leukotriene modulators, long-acting inhaled beta-2 agonists, theophylline, or the like. However, asthma of which symptoms are uncontrollable even after all of these general asthma medications have been used is called ‘severe asthma’. In addition, patients who have frequent asthma exacerbations or whose symptoms are not controlled despite maximal use of treatment, such as shortness of breath and cough or phlegm when oral steroids are stopped, are clinically classified as severe asthma patients. According to the guidelines of the American Thoracic Society (ATS) or the American or European Respiratory Society, severe asthma may be sometimes defined when asthma exacerbations occur more than twice a year despite the use of medications.

As described above, severe asthma without a clear diagnosis is difficult to define in one word because about 10% of asthma patients in Korea suffer from the severe asthma, and the degree of symptom control varies widely even among severe asthma patients.

In the case of eosinophilic asthma, a type of severe asthma, a high blood eosinophil level (150 cells/μL or more) appears as a symptom. Eosinophils are a type of granulocytic leukocyte that has eosinophilic granules in the cytoplasm and are major cells participating in allergic reactions Small granules in the cytoplasm of eosinophils contain several chemical mediators such as peroxidase, RNase, DNase, lipolytic enzyme, plasminogen, and the like, and the mediators are secreted by the degranulation process according to the activation of eosinophils and destroy both parasites and surrounding tissues.

There is no specific blood test for eosinophilic asthma, and it is common to count the number of eosinophils through a blood test.

On the other hand, Raman analysis is a useful measurement method for detecting chemical species (type of protein, lipid, RNA, DNA, etc.) of a sample. This is because organic and inorganic molecules have their own Raman shift spectrum. However, there is too much eosinophil information in the Raman spectrum, so it is difficult to classify the data of the Raman spectrum.

### SUMMARY

Embodiments of the inventive concept provide a method and device for processing Raman data of eosinophils based on artificial intelligence, which quickly perform classification as to whether eosinophils is normal or abnormal by measuring eosinophils with Raman equipment and comparing the chemical and optical properties of the eosinophils for accurate diagnosis of eosinophilic asthma, and provide a diagnostic basis for eosinophil-diseases

However, problems to be solved by the inventive concept may not be limited to the above-described problems. Although not described herein, other problems to be solved by the inventive concept can be clearly understood by those skilled in the art from the following description.

According to an embodiment, a method for processing Raman data of eosinophil based on artificial intelligence includes generating Raman data by performing Raman analysis using a specific wavelength on eosinophils isolated from blood of a diagnosed person, pre-processing the generated Raman data, assigning a weight to the pre-processed Raman data for each of components including a nucleus, a cell membrane, a granule, and a background, classifying data for each of the components based on a result of assigning the weight, extracting data in which the component is the granule based on a classified result, and determining whether a specific disease has occurred in the diagnosed person through eosinophil characteristics of the diagnosed person based on the extracted data.

The Raman data may be data in which two-dimensional data mapped for each point of the specific wavelength is arranged in an order corresponding to a traveling direction of the specific wavelength, and the two-dimensional data mapped for each point may include different Raman spectra.

The assigning of the weight may include converting the two-dimensional data mapped for each point into one-dimensional data by arranging the two-dimensional data in a line, extracting representative data for each of the components from the converted one-dimensional data, and assigning a Raman spectrum at a point corresponding to each extracted representative data as a weight for the each extracted representative data.

The classifying of the data may include performing cluster classification for each of the components using k-means clustering, and the extracting of the representative data may include performing data labeling by giving different labels to the components, determining whether a label labeled in the data is a label corresponding to the granule, and extracting only data labeled with a label corresponding to the granule among the labeled data, based on a result of the determination.

The extracted data may be Raman data in which the component is the granule among the pre-processed Raman data.

The determining of whether the specific disease has occurred may include analyzing the extracted data using an artificial intelligence-based first model and determining whether eosinophil characteristics of the diagnosed person are within a normal range or an abnormal range to determine whether the specific disease has occurred.

The first model may be built by learning a first Raman data processing process for a plurality of eosinophils isolated from the blood of a plurality of existing patients with the specific disease, a second Raman data processing process for a plurality of eosinophils isolated from the blood of a plurality of normal people without the specific disease, the eosinophil characteristics of the plurality of existing patients acquired by performing the first Raman data processing process and the eosinophil characteristics of the plurality of normal people acquired by performing the second Raman data processing process.

The method may further include generating an artificial intelligence-based second model using learning data for a plurality of existing patients, and predicting a cause of occurrence of the specific disease of a new patient by applying the granule data of the new patient determined as having the specific disease to the second model. The learning data may include granule data extracted through Raman analysis on eosinophils isolated from blood of each of the plurality of existing patients as input data, and data indicating whether or not each of the plurality of existing patients has the specific disease, as output data.

The predicting of the cause of occurrence of the specific disease may include classifying an activation level of eosinophilic granule and a type of proteins constituting the eosinophilic granule based on the granule data of the new patient, and predicting a cause of occurrence of the specific disease for the new patient based on the classified activation level and the type of proteins.

The input data may further include treatment and treatment result data of each of the plurality of existing patients, and the second model may be trained by performing labeling for each cause of the specific disease using the treatment and treatment result data.

The predicting of the cause of occurrence of the specific disease may include classifying the cause of the specific disease diagnosed in the new patient into at least one label among a plurality of labels, and the plurality of labels may include congenital, secondary, primary and idiopathic.

The input data may further include gender and age data of each of the plurality of existing patients, and the second model may have learned symptom levels of the specific disease by gender and age using the gender and age data.

The predicting of the cause of occurrence of the specific disease may include predicting a likelihood that a symptom of the specific disease diagnosed in the new patient worsens based on gender and age of the new patient.

The input data further may include granule data of a patient who has been cured of the specific disease or granule data of a normal person without the specific disease.

In addition, another method and another system, and a computer-readable recording medium for recording a computer program for executing the method may be further provided.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a schematic block diagram of a device for processing Raman data of eosinophil based on based on artificial intelligence according to the inventive concept;

FIG. 2 is a flowchart of a method for processing Raman data of eosinophil based on artificial intelligence according to the inventive concept.

FIG. 3 is a diagram for describing Raman data according to the inventive concept.

FIG. 4 is a diagram for describing weight assignment for each component according to the inventive concept.

FIG. 5 is a diagram for describing data extraction through cluster classification and data labeling for each component according to the inventive concept.

FIG. 6 is a diagram for describing determination of whether a diagnosed person is normal or not based on the eosinophil characteristics of the subject according to the inventive concept.

FIG. 7 is a schematic block diagram of an artificial intelligence-based eosinophilia prediction device according to the inventive concept.

FIG. 8 is a view for describing a prediction process performed by the artificial intelligence-based eosinophilia prediction device according to the inventive concept.

FIG. 9 is a view for describing a method for extracting granule data for prediction of eosinophilia according to the inventive concept.

FIG. 10 is a flowchart of an artificial intelligence-based eosinophilia prediction method according to the inventive concept.

### DETAILED DESCRIPTION

Advantages and features of the inventive concept and methods for achieving them will be apparent with reference to embodiments described below in detail in conjunction with the accompanying drawings. However, the inventive concept is not limited to the embodiments disclosed below, but can be implemented in various forms, and these embodiments are to make the disclosure of the inventive concept complete, and are provided so that this disclosure will be thorough and complete and will fully convey the scope of the invention to those of ordinary skill in the art, which is to be defined only by the scope of the claims.

The terminology used herein is for the purpose of describing particular embodiments only and is not intended to be limiting of the inventive concept. The singular expressions include plural expressions unless the context clearly dictates otherwise. In this specification, the terms “comprises” and/or “comprising” are intended to specify the presence of stated elements, but do not preclude the presence or addition of elements. Like reference numerals refer to like elements throughout the specification, and “and/or” includes each and all combinations of one or more of the mentioned elements. Although “first”, “second”, and the like are used to describe various components, these components are of course not limited by these terms. These terms are only used to distinguish one component from another. Thus, a first element discussed below could be termed a second element without departing from the teachings of the inventive concept.

Unless otherwise defined, all terms (including technical and scientific terms) used herein have the same meaning as commonly understood by one of ordinary skill in the art to which this invention belongs. It will be further understood that terms such as those defined in commonly used dictionaries, will not be interpreted in an idealized or overly formal sense unless expressly so defined herein.

Spatially relative terms, such as “below”, “beneath”, “lower”, “above”, and “upper” and the like, may be used herein for ease of description to describe one element or feature's relationship to another element(s) or feature(s) as illustrated in the figures. It will be understood that the spatially relative terms are intended to encompass different orientations of elements in use or operation in addition to the orientation depicted in the figures. For example, if an element in the figures is turned over, the element described as “below” or “beneath” the other element or features would then be oriented “above” the other element or features. Thus, the exemplary term “below” can encompass both an orientation of above and below. The element may be otherwise oriented and the spatially relative terms used herein interpreted accordingly.

Hereinafter, embodiments of the present disclosure will be described with reference to the accompanying drawings.

Before the description, the meaning of the terms used herein will be briefly described. However, it should be noted that the description of the terms is for the purpose of helping the understanding of the present specification, and is not used to limit the technical idea of the inventive concept unless explicitly described as limiting the inventive concept.

In the present specification, ‘learning model’ and ‘classification model’ are models built based on artificial intelligence, and may be trained based on various artificial intelligence algorithms. For example, all algorithms for learning such as Convolution Neural Network (CNN), Deep Neural Network (DNN), Recurrent Neural Network (RNN), K-Nearest Neighbors (KNN), and support vector machine (SVM) are applicable. That is, the ‘classification model’ and the ‘learning model’ described herein may be interpreted as the same meaning.

In the present specification, term ‘device’ includes various devices capable of providing a result to a user by performing arithmetic processing. For example, the device may be in the form of a computer and a mobile terminal. The computer may be in the form of a server that receives a request from a client and performs information processing. In addition, the computer may correspond to a sequencing device that performs sequencing. The mobile terminal may include a mobile phone, a smart phone, a personal digital assistant (PDA), a portable multimedia player (PMP), a navigation system, a notebook PC, a slate PC, a tablet PC, an ultrabook, and a wearable device, for example, a watch-type terminal (smart watch), a glass-type terminal (smart glass), a head mounted display (HMD), and the like.

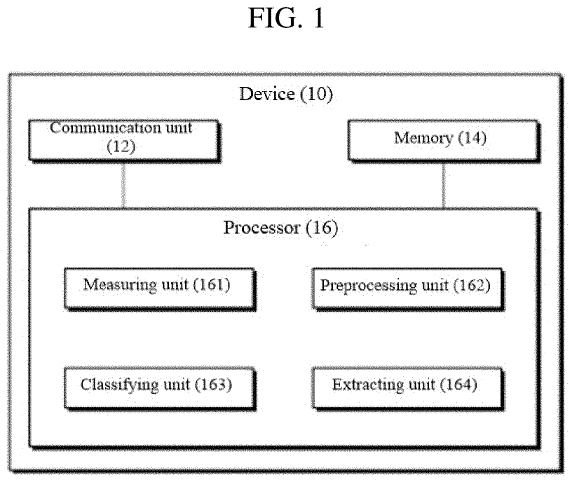

FIG. 1 is a schematic block diagram of a device for processing Raman data of eosinophils based on artificial intelligence according to the inventive concept.

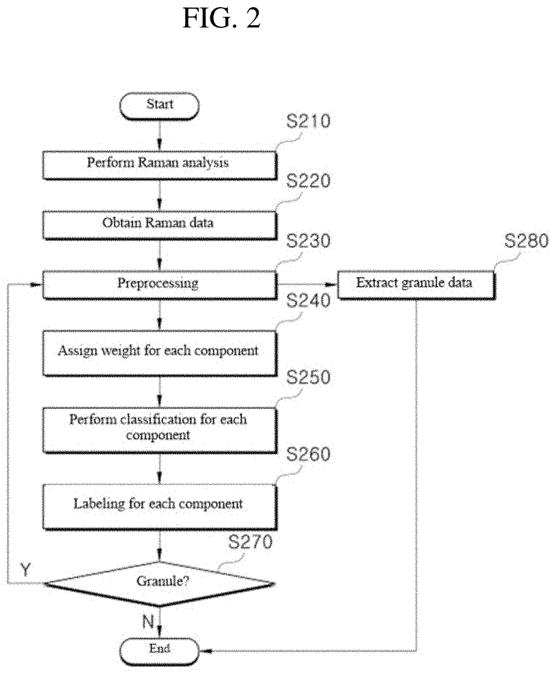

FIG. 2 is a flowchart of a method for processing Raman data of eosinophils based on artificial intelligence according to the inventive concept.

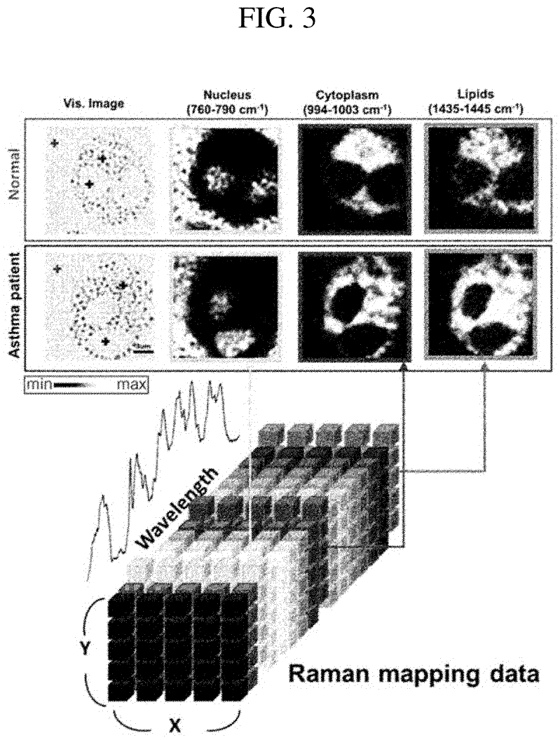

FIG. 3 is a diagram for describing Raman data according to the inventive concept.

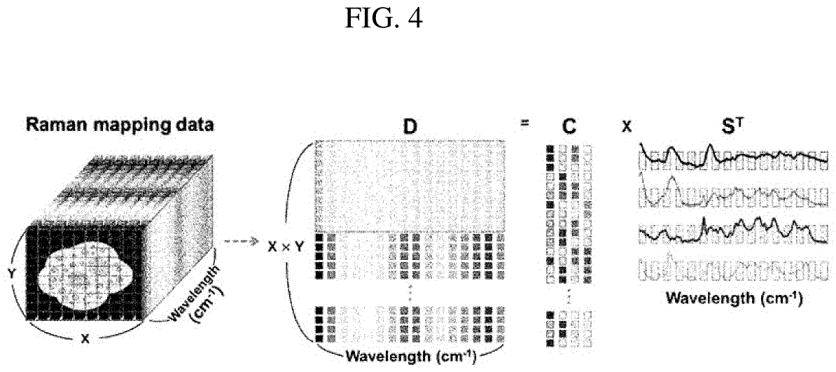

FIG. 4 is a diagram for describing weight assignment for each component according to the inventive concept.

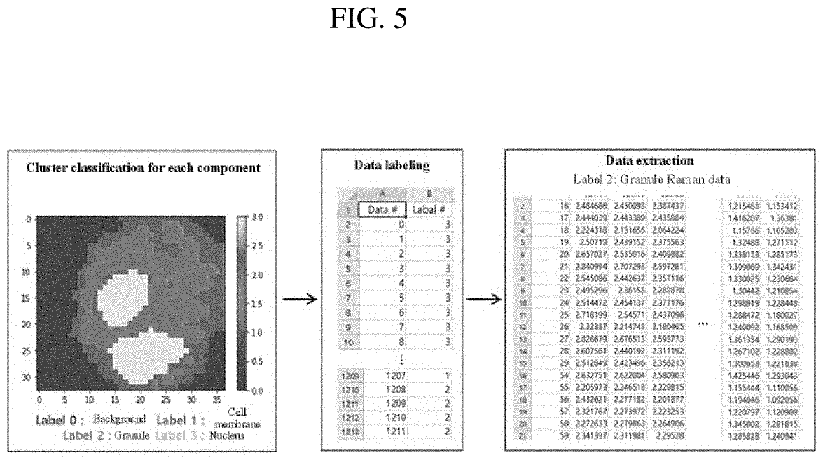

FIG. 5 is a diagram for describing data extraction through cluster classification and data labeling for each component according to the inventive concept.

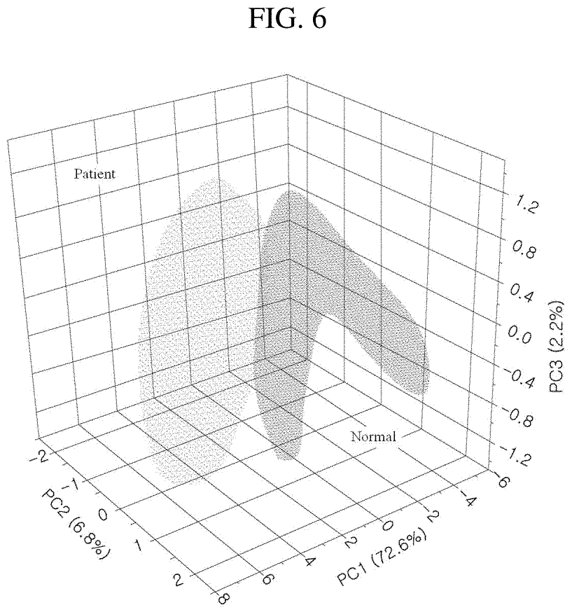

FIG. 6 is a diagram for describing the determination of whether the diagnosed person is normal or not based on the eosinophil characteristics of the diagnosed person according to the inventive concept.

Hereinafter, a device 10 for processing Raman data of eosinophil based on artificial intelligence (hereinafter referred to as a device) according to the inventive concept will be described.

The device 10 according to the inventive concept may fix eosinophils isolated from the blood of a subject in a petri dish, and then obtain Raman data including various components such as nucleus, cytoplasm, and background through Raman analysis.

The device 10 may perform preprocessing, such as normalization, on the obtained Raman data.

The device 10 may assign a weight to the preprocessed Raman data for each component included in the Raman data using an artificial intelligence-based learning model, perform classification for each component, and label the classified data to extract only data of granules among eosinophils.

In this case, the extracted data of granules is pre-processed data, and is data without separate processing and loss, to make it possible to compare the chemical properties of eosinophils.

The device 10 may include all of a variety of devices capable of providing a result to a user by performing arithmetic processing.

Here, the device 10 may be in the form of a computer. More specifically, the computer may include all of a variety of devices capable of providing a result to a user by performing arithmetic processing.

For example, the computer may include a desktop PC, a notebook as well as a smart phone, a tablet PC, a cellular phone, a PCS (Personal Communication Service) phone, a mobile terminal of a synchronous/asynchronous International Mobile Telecommunication-2000 (IMT-2000), a Palm Personal Computer (PC), a Personal Digital Assistant (PDA), and the like. Further, when a head mounted display (HMD) device includes a computing function, the HMD device may be the computer.

The device 10 may include a communication unit 12, a memory 14, and a processor 16. The processor 16 may include a measuring unit 161, a preprocessing unit 162, a classifying unit 163, and an extracting unit 164. Here, the device 10 may include fewer components or more components than the components shown in FIG. 1.

The communication unit 12 may include more than one module that enables wireless communication between the device 10 and an external device (not shown), between the device 10 and an external server (not shown), or between the device 10 and a communication network (not shown).

Here, the communication network (not shown) may transmit/receive various information among the device 10, an external device (not shown), and an external server (not shown). The communication network (not shown) may use various types of communication networks, for example, wireless communication schemes including WLAN (Wireless LAN), Wi-Fi, Wibro, Wimax, HSDPA (High Speed Downlink Packet Access), and the like, or wired communication schemes including Ethernet, xDSL (ADSL, VDSL), HFC (Hybrid Fiber Coax), FTTC (Fiber to The Curb), FTTH (Fiber To The Home) and the like.

On the other hand, the communication network (not shown) is not limited to the communication schemes presented above, and may include all types of communication schemes widely known or to be developed in the future in addition to the above-described communication schemes.

The communication unit 12 may also include one or more modules that connect the device 10 to one or more networks.

The memory 14 may store data that supports various functions of the device 10. The memory 14 may store a plurality of application programs (or applications) running on the device 10, at least one process for the operation of the device 10, an artificial intelligence-based learning model, data, and instructions. In addition, at least some of these application programs may exist for basic functions of the device 10. Meanwhile, the application program may be stored in the memory 14, installed on the device 10, and driven by the processor 16 to perform an operation (or function) of the device 10.

The processor 16 may generally control the overall operation of the device 10 as well as the operation related to the application program. The processor 16 may provide or process information or a function appropriate to a user by processing signals, data, information, and the like, which are input or output through the above-described components, or by executing an application program stored in the memory 14.

In addition, the processor 16 may control at least some of the components described with reference to FIG. 1 in order to execute an application program stored in the memory 14. In addition, the processor 16 may operate at least two or more of the components included in the device 10 in a combination thereof to execute the application program.

Also, the measuring unit 161, the preprocessing unit 162, the classifying unit 163, and the extracting unit 164 included in the processor 16 may perform respective functions as described above.

The measuring unit 161 may perform Raman analysis on eosinophils isolated from the blood of a diagnosed person. The measuring unit 161 may be a Raman spectrometer for Raman analysis, but is not limited thereto. According to an embodiment, the measuring unit 161 may be configured as a separate device from the device 10 to transmit/receive data through a communication module, or may be configured to be included in the device 10. The measuring unit 161 may measure a Raman spectrum that appears differently at the point of a specific wavelength according to different amounts of energy change of components of the eosinophil when laser light with a specific wavelength is incident on the eosinophil. Here, the specific wavelength may be a preset wavelength.

The Raman data generated according to the measurement by the measuring unit 161 is pre-processed by the preprocessing unit 162, such as normalization.

The classifying unit 163 may perform cluster classification for each component by giving a weight to the preprocessed Raman data for each component.

The extracting unit 164 may label data classified for each component and extract only data whose label is granules.

Although it has been described that a plurality of components of the processor 16 perform their functions respectively, the operation of the processor 16 is described in such a manner that the processor 16 is divided into components by functions for convenience of description. That is, the processor 16 is able to perform all of the above operations, and only when the measuring unit 161 is composed of a separate device from the device 10, the processor 16 may receive Raman data from the measuring unit 161 that is a separate device.

Hereinafter, a method for processing Raman data of eosinophils based on artificial intelligence in the processor 16 will be described with reference to FIGS. 2 to 6. Here, the operation of the processor 16 may be performed by the device 10.

Referring to FIG. 2, the processor 16 may perform Raman analysis using a specific wavelength on eosinophils isolated from the blood of a diagnosed person (S210). The processor 16 may generate Raman data based on an analyzed result (S220). A detailed description of step S210 will be omitted because it overlaps the above description.

The Raman analysis used in the inventive concept may be spectroscopic analysis for measuring inelastic scattering of light particles of an incident laser, but is not limited thereto.

The Raman data obtained through Raman analysis in step S210 may be data in which two-dimensional data mapped to each point of a specific wavelength are arranged in an order corresponding to the traveling direction of the specific wavelength.

Referring to FIG. 3, a plurality of two-dimensional data having a horizontal length of X and a vertical length of Y may be arranged according to a traveling direction of a wavelength to generate Raman data (Raman mapping data).

As shown in FIG. 3, two-dimensional data mapped to each point of the wavelength may have different components clearly expressed in an image. This is because the optical properties of the components are different at each point. It can be seen that the two-dimensional data mapped to the point of a wavelength of 760-790 cm⁻¹ reveals relatively clearly the shape of the nucleus among the components.

In addition, the two-dimensional data mapped for each point may include different Raman spectra. As described above, different Raman spectra are measured for each point of a wavelength according to different amounts of energy change for each component, and the Raman spectrum measured for each point may be included in the two-dimensional data mapped for each point.

Although it is descried that the Raman data is generated by including the Raman spectrum in the two-dimensional data mapped for each point, the inventive concept is not limited thereto. According to an embodiment, the Raman data may be generated in such a way that the two-dimensional data and the Raman spectrum are associated for each point of the wavelength.

Referring back to FIG. 2, the processor 16 may preprocess the generated Raman data (S230).

Specifically, the processor 16 may normalize the generated Raman data. Since data normalization is well-known, a detailed description thereof will be omitted.

Steps S240 and S280 described below may be performed using a pre-established artificial intelligence-based learning model. A description of the learning model will be given later.

The processor 16 may assign weights to components of the preprocessed Raman data, the components including nucleus, cell membranes, granules, and backgrounds (S240).

Specifically, the processor 16 may convert two-dimensional data mapped to points into one-dimensional data by arranging the two-dimensional data in a line.

Referring to FIG. 4, the Raman data formed as the two-dimensional data for each point of a wavelength is converted into one-dimensional data for each point.

Thereafter, the processor 16 may extract representative data for each of the components from the converted one-dimensional data. Here, the representative data may mean data that best represents a pattern of optical properties of each component.

That is, referring to FIG. 4, among the one-dimensional data mapped for each point, data that best represents the pattern of optical properties of the nucleus among components may be extracted as representative data for the nucleus, data that best represents the pattern of optical properties of the cell membrane may be extracted as representative data for the cell membrane, data that best represents the pattern of optical properties of the granules may be extracted as the representative data for the granules, and data that best represents the pattern of optical properties of the background may be extracted as the representative data for the background.

Thereafter, the processor 16 may assign a Raman spectrum at a point corresponding to each extracted representative data as a weight for the each extracted representative data.

That is, referring to FIG. 4, a Raman spectrum measured at each point to which the extracted representative data is mapped may be applied to the representative data as a weight. As described above, by amplifying representative data representing a pattern for each component by weighting the representative data, it is possible to increase the accuracy of classification for each component thereafter.

Referring back to FIG. 2, the processor 16 may classify data for each component based on the weighted result (S250).

Specifically, the processor 16 may perform cluster classification for each component using k-means clustering. That is, a central point may be clustered for each component. Here, the central point for each component may be set based on representative data for each component to which the weight is assigned, but is not limited thereto.

Referring to FIG. 5, the processor 16 may represent the background as label 0, the cell membrane as label 1, the granules as label 2, and the nucleus as label 3 to perform cluster classification.

Referring back to FIG. 2, the processor 16 may extract data in which the component is the granule based on the classified result (S280).

Specifically, the processor 16 may perform data labeling by assigning different labels to components (S260). Thereafter, the processor 16 may determine whether the label of labeled data is a label corresponding to the granule (S270). Thereafter, the processor 16 may extract only data labeled with a label corresponding to the granule from among the labeled data, based on a result of the determination.

As shown in FIG. 5, data labeling may be performed on result data with an image form generated as the cluster classification has been completed in step S250 for each pixel of the image. In this case, data labeling may be performed based on a label assigned when performing cluster classification.

When the entire data labeling for the image is completed in this way, the processor 16 may determine whether the label of the data is a label corresponding to the granule, and extract only data determined as the label corresponding to the granule.

Here, the extracted data may be Raman data in which the component is the granule among the pre-processed Raman data. In this way, it is possible to compare the chemical properties for each eosinophil by finally extracting pre-processed data and utilizing data without separate processing and loss.

Although it has been described that step S280 includes steps S260 and S270, the inventive concept is not limited thereto and steps S260 to S280 may be sequentially performed as individual operations.

Further, although not shown in FIG. 2, when the data in which the component is granule is extracted, the processor 16 may determine whether a specific disease has occurred in the diagnosed person through the eosinophil characteristics of the diagnosed person based on the extracted data.

Here, the specific disease may be eosinophilic asthma, but is not limited thereto, and may include all of various diseases accompanied by an increase in eosinophils.

As descried above, by extracting only the data on granules among the components of eosinophils, the processor 16 may determine whether the diagnosed person is normal or abnormal for the specific disease with only the chemical characteristics of the granules in question among the chemical characteristics of eosinophils.

Specifically, the processor 16 may analyze the extracted data using an artificial intelligence-based learning model and determine whether the eosinophil characteristic of the diagnosed person is within a normal range or an abnormal range to determine whether a specific disease has occurred.

Here, the learning model may be built by learning a first Raman data processing process for a plurality of eosinophils isolated from the blood of a plurality of patients with a specific disease, a second Raman data processing process for a plurality of eosinophils isolated from the blood of a plurality of normal people without the specific disease, the eosinophil characteristics of the plurality of patients acquired by performing the first Raman data processing process and the eosinophil characteristics of the plurality of normal people acquired by performing the second Raman data processing process.

That is, the learning model may accurately grasp the difference in eosinophil characteristics between a normal person and a patient by performing learning while comparing various data of the normal people for a specific disease with various data of the patients for the specific disease. As shown in FIG. 6, the eosinophil characteristics of the normal person based on the Raman data of granules, extracted from eosinophils of the normal people are clearly different from the eosinophil characteristics of the patients based on the Raman data of granules extracted from eosinophils of the patients.

Therefore, the learning model may determine whether the eosinophil characteristic of the diagnosed person falls within the normal range or the abnormal range by learning the differences, and when it is determined that the eosinophil characteristic falls within the abnormal range, predict that the specific disease has occurred in the diagnosed person.

It is described that the granule data of eosinophils is extracted after isolating eosinophils from the blood of the diagnosed person, but the inventive concept is not limited thereto, and related diseases may be predicted by applying the granule data extraction algorithm not only to eosinophils but also to other granular cells.

It is described with reference to FIGS. 1 to 6 that the device 10 of the inventive concept determines whether or not an eosinophil-related disease has occurred in a diagnosed person through Raman data processing.

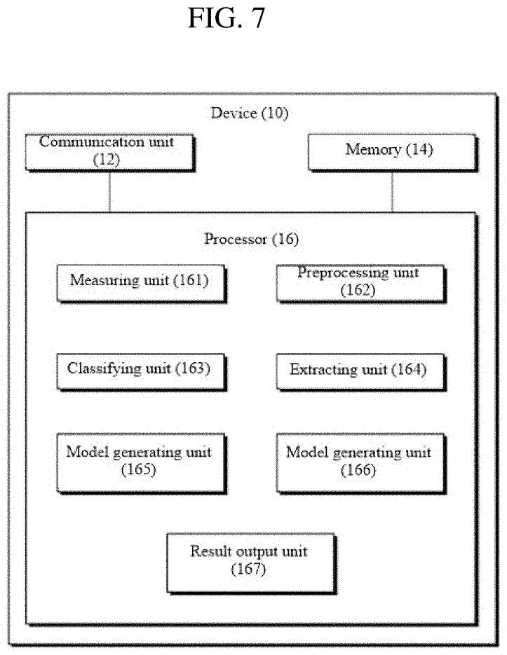

Hereinafter, it will be described with reference to FIGS. 7 to 10 that the device 10 of the inventive concept not only determines whether an eosinophil-related disease has occurred in a diagnosed person but also predicts the cause of the occurrence of an eosinophil-related disease (hereinafter, eosinophilia) in the diagnosed person.

FIG. 7 is a schematic block diagram of an artificial intelligence-based eosinophilia prediction device according to the inventive concept.

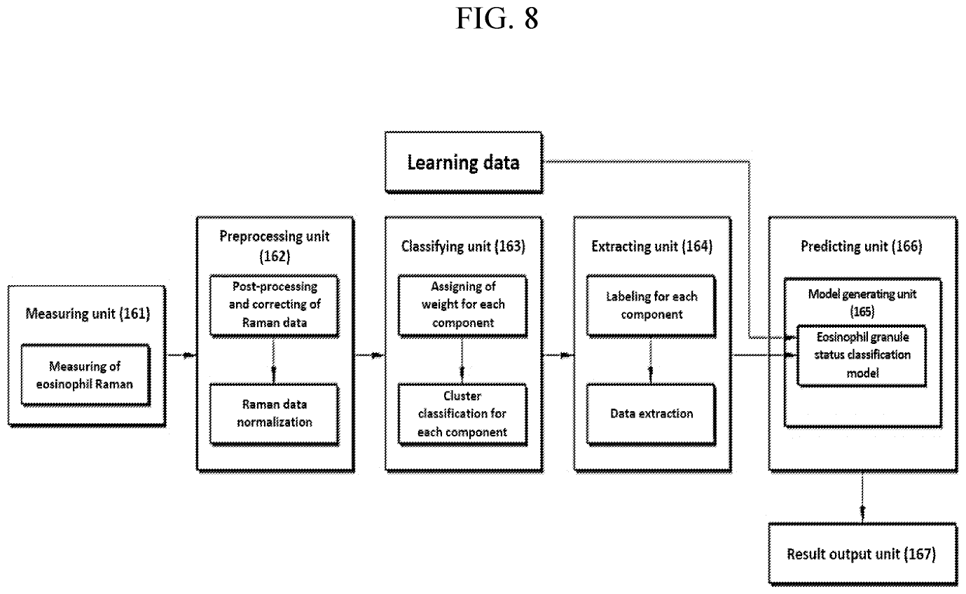

FIG. 8 is a view for describing a prediction process performed by the artificial intelligence-based eosinophilia prediction device according to the inventive concept.

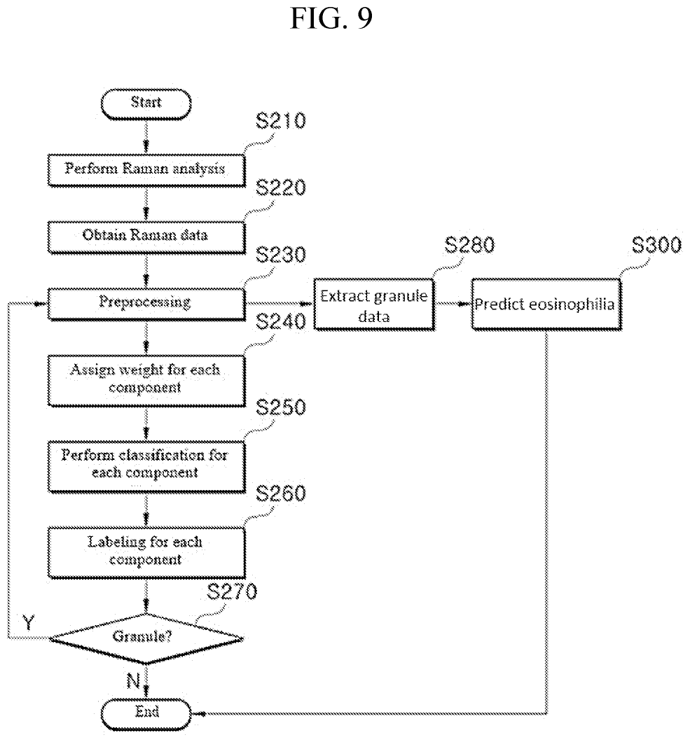

FIG. 9 is a view for describing a method for extracting granule data for prediction of eosinophilia according to the inventive concept.

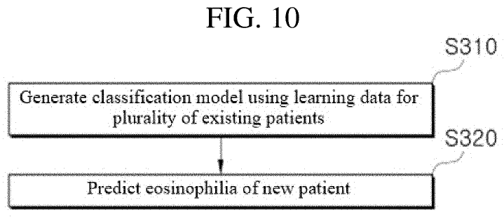

FIG. 10 is a flowchart of an artificial intelligence-based eosinophilia prediction method according to the inventive concept.

Hereinafter, an artificial intelligence-based eosinophilia prediction device 10 (hereinafter referred to as a device) according to the inventive concept will be described with reference to FIG. 7.

Referring to FIG. 7, the device 10 may include the communication unit 12, the memory 14, and the processor 16. Here, the device 10 may include fewer components or more components than the components shown in FIG. 7.

The communication unit 12 may include more than one module that enables wireless communication between the device 10 and an external device (not shown), between the device 10 and an external server (not shown), or between the device 10 and a communication network (not shown).

Here, the communication network (not shown) may transmit/receive various information among the device 10, an external device (not shown), and an external server (not shown). The communication network (not shown) may use various types of communication networks, for example, wireless communication schemes including WLAN (Wireless LAN), Wi-Fi, Wibro, Wimax, HSDPA (High Speed Downlink Packet Access), and the like, or wired communication schemes including Ethernet, xDSL (ADSL, VDSL), HFC (Hybrid Fiber Coax), FTTC (Fiber to The Curb), FTTH (Fiber To The Home) and the like.

On the other hand, the communication network (not shown) is not limited to the communication schemes presented above, and may include all types of communication schemes widely known or to be developed in the future in addition to the above-described communication schemes.

The communication unit 12 may also include one or more modules that connect the device 10 to one or more networks.

The memory 14 may store data that supports various functions of the device 10. The memory 14 may store a plurality of application programs (or applications) running on the device 10, at least one process for the operation of the device 10, AI-based classification models, data, and instructions. In addition, at least some of these application programs may exist for basic functions of the device 10. Meanwhile, the application programs may be stored in the memory 14, installed on the device 10, and driven by the processor 16 to perform an operation (or function) of the device 10.

The processor 16 may generally control the overall operation of the device 10 as well as the operation related to the application program. The processor 16 may provide or process information or a function appropriate to a user by processing signals, data, information, and the like, which are input or output through the above-described components, or by executing an application program stored in the memory 14.

In addition, the processor 16 may control at least some of the components described with reference to FIG. 7 in order to execute an application program stored in the memory 14. In addition, the processor 16 may operate at least two or more of the components included in the device 10 in a combination thereof to execute the application program.

Referring to FIG. 7, the processor 16 of the device 10 may include the measuring unit 161, the preprocessing unit 162, the classifying unit 163, the extracting unit 164, a model generating unit 165, a predicting unit 166, and a result output unit 167. Here, the processor 16 may include fewer components or more components than the components shown in FIG. 7.

The model generating unit 165 of the device 10 according to the inventive concept may generate a classification model using learning data for a plurality of existing patients. Here, the classification model may be the same as the learning model described above, but is not limited thereto, and according to embodiments, the learning model may be a model for predicting whether or not a disease occurs, and the classification model may be a model for predicting the cause of occurrence of a disease.

Here, the existing patients may mean patients who have been diagnosed with eosinophilia and are being treated, and the learning data is data used for training the classification model, and may include various basic data related to the patients.

The model generating unit 165 may be built by learning the granule data of the existing patient. A detailed description thereof will be provided later.

The predicting unit 166 of the device 10 according to the inventive concept may predict whether a new patient has eosinophilia by applying the granule data of the new patient to the classification model.

Here, the new patient is a patient showing asthma symptoms, and the predicting unit 166 may determine whether the new patient is a general asthma patient or a patient showing asthma symptoms due to an increase in eosinophils through the classification model. A detailed description thereof will be provided later.

As described above, granule data of existing patients is needed to generate the classification model, and granule data of the new patient is needed to predict whether or not eosinophilia has occurred. Here, the granule data may be data extracted by performing Raman analysis on eosinophils isolated from the blood of a patient (existing patient, new patient).

Referring to FIG. 8, the measuring unit 161 of the device 10 may perform Raman analysis on eosinophils isolated from the patient's blood. The measuring unit 161 may be a Raman spectrometer for Raman analysis, but is not limited thereto. According to an embodiment, the measuring unit 161 may be configured as a separate device from the device 10 to transmit/receive data through a communication module, or may be configured to be included in the device 10. The measuring unit 161 may measure a Raman spectrum that appears differently at the point of a specific wavelength according to different amounts of energy change of components of the eosinophil when laser light with a specific wavelength is incident on the eosinophil. Here, the specific wavelength may be a preset wavelength.

The Raman data generated according to the measurement by the measuring unit 161 may be preprocessed through normalization in the preprocessing unit 162.

The classifying unit 163 may perform cluster classification for each component by giving a weight to the preprocessed Raman data for each component.

The extracting unit 164 may label data classified for each component and extract only data whose label is granules.

The model generating unit 165 may be built by learning the granule data of existing patients extracted through the measuring unit 161, the preprocessing unit 162, the classifying unit 163, and the extracting unit 164.

The predicting unit 166 may determine whether or not the new patient has eosinophilia by applying, to the classification model generated by the model generating unit 165, the granule data of the new patient extracted through the measuring unit 161, the preprocessing unit 162, the classifying unit 163, and the extracting unit 164.

The result output unit 167 may output a diagnosis result predicted by the predicting unit 166. The result output unit 167 may include a display unit. The display unit may implement a touch screen in such a manner that the display unit forms a layer structure with or is integrally formed with a touch sensor. Such a touch screen may function as a user input unit that provides an input interface between the device 10 and the user and may provide an output interface between the device 10 and the user at the same time.

Although it has been described that a plurality of components of the processor 16 perform their functions respectively, the operation of the processor 16 is described in such a manner that the processor 16 is divided into components by functions for convenience of description. That is, the processor 16 is able to perform all of the above operations, and only when the measuring unit 161 is composed of a separate device from the device 10, the processor 16 may receive Raman data from the measuring unit 161 that is a separate device.

Hereinafter, with reference to FIGS. 9 and 10, a method for isolating eosinophils from a patient's blood, performing Raman analysis, and extracting granule data through this in the processor 16 of the inventive concept will be described in detail. Here, the operation of the processor 16 may be performed by the device 10.

Referring to FIG. 9, the measuring unit 161 of the processor 16 may perform Raman analysis using a specific wavelength on eosinophils isolated from the blood of a patient (S210). The processor 16 may generate Raman data based on an analyzed result (S220). In this case, the Raman analysis used in the inventive concept may be spectroscopic analysis for measuring inelastic scattering of light particles of an incident laser, but is not limited thereto.

Specifically, after fixing eosinophils isolated from a patient's blood in a petri dish, Raman data including at least one component of a nucleus, cytoplasm, and background may be obtained through Raman analysis.

The Raman data obtained through Raman analysis in step S210 and step S220 may be data in which two-dimensional data mapped to each point of a specific wavelength are arranged in an order corresponding to the traveling direction of the specific wavelength.

As described above, referring to FIG. 3, a plurality of two-dimensional data having a horizontal length of X and a vertical length of Y may be arranged according to a traveling direction of a wavelength to generate Raman data (Raman mapping data).

As shown in FIG. 3, two-dimensional data mapped to each point of the wavelength may have different components clearly expressed in an image. This is because the optical properties of the components are different at each point. It can be seen that the two-dimensional data mapped to the point of a wavelength of 760-790 cm⁻¹ reveals relatively clearly the shape of the nucleus among the components.

In addition, the two-dimensional data mapped for each point may include different Raman spectra. As described above, different Raman spectra are measured for each point of a wavelength according to different amounts of energy change for each component, and the Raman spectrum measured for each point may be included in the two-dimensional data mapped for each point.

Although it is descried that the Raman data is generated by including the Raman spectrum in the two-dimensional data mapped for each point, the inventive concept is not limited thereto. According to an embodiment, the Raman data may be generated in such a way that the two-dimensional data and the Raman spectrum are associated and mapped for each point of the wavelength.

Referring back to FIG. 9, the preprocessing unit 162 of the processor 16 may preprocess the generated Raman data (S230). Specifically, the processor 16 may normalize the generated Raman data. Since data normalization is well-known, a detailed description thereof will be omitted.

The processor 16 may assign a weight to the preprocessed Raman data for each of components included in the Raman data using an artificial intelligence-based learning model, perform classification for each component, and label the classified data to extract only data of granules among eosinophils. In this case, the extracted data of granules is pre-processed data, and is data without separate processing and loss, to make it possible to compare the chemical properties of eosinophils. Details will be described below.

The classifying unit 163 of the processor 16 may assign weights to components of the preprocessed Raman data, the components including nucleus, cell membranes, granules, and backgrounds (S240).

Specifically, the classifying unit 163 may convert two-dimensional data mapped for each point into one-dimensional data by arranging the two-dimensional data in a line.

As described above, referring to FIG. 4, the Raman data formed as the two-dimensional data for each point of a wavelength is converted into one-dimensional data for each point.

Thereafter, the classifying unit 163 may extract representative data for each component from the converted one-dimensional data. Here, the representative data may mean data that best represents a pattern of optical properties of each component.

That is, referring to FIG. 4, among the one-dimensional data mapped for each point, data that best represents the pattern of optical properties of the nucleus among components may be extracted as representative data for the nucleus, data that best represents the pattern of optical properties of the cell membrane may be extracted as representative data for the cell membrane, data that best represents the pattern of optical properties of the granules may be extracted as the representative data for the granules, and data that best represents the pattern of optical properties of the background may be extracted as the representative data for the background.

Thereafter, the classifying unit 163 may assign a Raman spectrum at a point corresponding to each extracted representative data as a weight for the each extracted representative data.

That is, referring to FIG. 4, a Raman spectrum measured at each point to which the extracted representative data is mapped may be applied to the representative data as a weight. As described above, by amplifying representative data representing a pattern for each component by weighting the representative data, it is possible to increase the accuracy of classification for each component thereafter.

Referring back to FIG. 9, the classifying unit 163 of the processor 16 may classify data for each component based on the weighted result (S250).

Specifically, the classifying unit 163 may perform cluster classification for each component using k-means clustering. That is, a central point may be clustered for each component. Here, the central point for each component may be set based on representative data for each component to which the weight is assigned, but is not limited thereto.

As described above, referring to FIG. 5, the classifying unit 163 may represent the background as label 0, the cell membrane as label 1, the granules as label 2, and the nucleus as label 3 to perform cluster classification.

Referring back to FIG. 9, the extracting unit 164 of the processor 16 may extract data (hereinafter, granule data) in which the component is the granule, based on the classified result.

Specifically, the extracting unit 164 may perform data labeling by assigning different labels to components (S260). Thereafter, the extracting unit 164 may determine whether the label of labeled data is a label corresponding to the granule (S270). Thereafter, the extracting unit 164 may extract only data labeled with a label corresponding to the granule from among the labeled data, based on a result of the determination (S280).

As shown in FIG. 5, data labeling may be performed on result data with an image form generated as the cluster classification has been completed in step S250 for each pixel of the image. In this case, data labeling may be performed based on a label assigned when performing cluster classification.

When the entire data labeling for the image is completed in this way, the extracting unit 164 may determine whether the label of the data is a label corresponding to the granule, and extract only data determined as the label corresponding to the granule.

Here, the extracted data may be Raman data in which the component is the granule among the pre-processed Raman data. In this way, it is possible to compare the chemical properties for each eosinophil by finally extracting pre-processed data and utilizing data without separate processing and loss. That is, FIG. 9 may mean extracting data which has been preprocessed in step S230 as final data for Raman data of which the component is granules, in S270, rather than mean returning to step S230 when the component is granules in step S270, and re-performing preprocessing.

The granule data extracted as described may be used to predict eosinophilia (S300). Hereinafter, step S300 will be described in detail.

Referring to FIG. 10, the model generating unit 165 of the processor 16 may generate a classification model using learning data for a plurality of existing patients (S310).

Here, the existing patient may mean a patient who has been diagnosed with eosinophilia and is being treated.

Here, the learning data is data used for training the classification model, and may include various basic data related to the patients.

The learning data may be a dataset including input data and output data.

The input data may be granule data extracted through Raman analysis of eosinophils isolated from the blood of each of the plurality of existing patients, and the output data may be data indicating whether or not each of the plurality of existing patients has eosinophilia. That is, the model generating unit 165 may analyze the eosinophil granule status (activation level of eosinophilic granules and types of proteins constituting eosinophilic granules) of each of the existing patients, and learn the activation level and the type of protein when eosinophilia has occurred.

The predicting unit 166 of the processor 16 may predict whether a new patient has eosinophilia by applying the granule data of the new patient to the classification model (S320).

Here, the new patient is a patient showing asthma symptoms, and the predicting unit 166 may determine whether the new patient is a general asthma patient or a patient showing asthma symptoms due to an increase in eosinophils through the classification model.

In one embodiment, the predicting unit 166 may classify the activation level of the eosinophilic granule and the type of protein constituting the eosinophilic granule based on the granule data of a new patient, and predict whether eosinophilia has occurred in the new patient based on the classified activation level and the type of protein.

That is, since the classification model had learned the activation level and the characteristics of the type of protein when eosinophilia has occurred, it is possible to analyze the new patient's granule data and determine whether eosinophilia has occurred in the new patient by identifying the activation level of the new patient's eosinophilic granules and the types of proteins constituting the eosinophilic granules.

In another embodiment, the predicting unit 166 may classify the activation level of the eosinophilic granule and the type of protein constituting the eosinophilic granule based on the granule data of the new patient, and predict whether eosinophilia occurs in the new patient and the cause of the occurrence of the eosinophilia, based on the classified activation level and the type of protein.

That is, the types and compositions of the proteins constituting the eosinophilic granules are different depending on the cause of eosinophilia. Therefore, the classification model learns the activation level and the characteristics of the protein type when eosinophilia occurs, and therefore identify that a specific protein is expressed in eosinophils at a specific activation level. Accordingly, the predicting unit 166 may analyze the new patient's granule data and determine whether eosinophilia has occurred in the new patient and the cause of the occurrence of the eosinophilia by identifying the activation level of the new patient's eosinophilic granules and the types of proteins constituting the eosinophilic granules.

In this case, the predicting unit 166 may determine the case of 500 or more eosinophils per 1 μL of peripheral blood as eosinophilia, and classify mild eosinophilia (500-1,500 cells/μL), moderate eosinophilia (1,500-5,000 cells)/μL), and severe eosinophilia (5,000 cells/μL) depending on the number of eosinophils.

According to an embodiment, the learning data of the inventive concept may further include treatment and treatment result data of each of the plurality of existing patients as input data.

The classification model generated in step S310 may be further trained by performing labeling for each cause of eosinophilia by further using the treatment and treatment result data.

Eosinophilia may be classified into congenital, secondary, primary and idiopathic. The congenital is classified as an autosomal dominant inheritance, the secondary is classified as cases including parasitic infections, allergic diseases, and adverse drug reactions (more specifically, viral/fungal infections, chronic inflammatory causes, chronic graft-versus-host disease, autoimmune diseases, adrenal insufficiency and solid tumors and some hematopoietic cell tumors), the primary is classified as cases of neoplastic and clonal, and the idiopathic is classified as cases other than secondary and primary.

As described above, the causes of eosinophilia have various disease or disorder, and accordingly, there are various treatments and treatment results thereof. Accordingly, the classification model of the inventive concept may learn treatment and treatment result data of existing patients having different causes of disease and different treatment and treatment results. Accordingly, the classification model may predict the cause of the disease when a specific activation level and a specific protein type are classified.

Further, in step S320, when the new patient is diagnosed as having eosinophilia, the predicting unit 166 may classify the cause of eosinophilia diagnosed in the new patient into at least one label among a plurality of labels. The plurality of labels may include congenital, secondary, primary and idiopathic.

That is, the predicting unit 166 may analyze the granule data of the new patient to classify the activation level of the eosinophilic granules of the new patient and the types of proteins constituting the eosinophilic granules, and determine the cause of occurrence of eosinophilia of the new patient by labeling the cause corresponding to the classified characteristics. For example, when the cause corresponding to the classified characteristics is secondary, the predicting unit 166 may label the cause of the new patient's onset as secondary.

According to an embodiment, the learning data of the inventive concept may further include the gender and age data of each of the plurality of existing patients as the input data.

The classification model generated in step S310 may learn the gender and age-specific symptom levels of eosinophilia by using the gender and age data.

Eosinophilia may be classified into mild eosinophilia (500-1,500 cells/μL), moderate eosinophilia (1,500-5,000 cells/μL), and severe eosinophilia 5,000 cells/μL) depending on the number of eosinophils, and symptoms of eosinophilia may differ depending on gender or age. That is, the classification model may learn the symptom level of eosinophilia in a specific gender and a specific age by further using the gender and age data.

When the new patient is diagnosed as having eosinophilia in step S320, the predicting unit 166 may predict the possibility of worsening of the symptoms of eosinophilia diagnosed in the new patient based on the gender and age of the new patient.

As an example of a patient whose the gender is female, the age is 60s and the symptom level represents moderate eosinophilia (1,500-5,000 cells/μL), when a new patient is diagnosed as eosinophilia having occurred as a result of analyzing the granule data of the new patient having the age of 60s and the number of eosinophils in the patient is 1400 cells/μL, the predicting unit 166 may predict that the patient's symptoms are likely to deteriorate from mild to moderate.

According to an embodiment, the learning data of the inventive concept may further include granule data of a patient who has been cured of eosinophilia or granule data of a normal person without a specific disease as the input data.

That is, according to the inventive concept, the classification model may be trained using granule data extracted through Raman analysis on eosinophils isolated from the blood of a patient who was diagnosed with eosinophilia and cured, as a control group (normal person). In addition, the classification model may be trained using granule data extracted through Raman analysis on eosinophils isolated from the blood of a normal person without a specific disease as a control group (normal person). As described above, it is possible to increase the prediction accuracy of the classification model by learning data of not only existing patients (patients diagnosed with eosinophilia and being treated) but also normal people.

The diagnosis result of the new patient predicted in step S320 may be output through the result output unit 167.

Although it is described with reference to FIGS. 2, 9, and 10 that steps are sequentially performed, this is merely illustrative of the technical idea of the embodiment. Those of ordinary skill in the art to which this embodiment belongs may perform various modifications and variations such as changing the order described in FIGS. 2, 9, and 10 or performing one or more of the plurality of steps in parallel without departing from the essential features of the present embodiment, so that the inventive concept is not limited to chronological order in FIGS. 2, 9, and 10.

The method according to an embodiment of the inventive concept described above may be implemented as a program (or application) to be executed in combination with a computer, which is hardware, and stored in a medium. Here, the computer may be the device 10 described above.

The above-described program may include codes coded in a computer language, such as C, C++, JAVA, or a machine language, which are readable by a processor (CPU) of the computer through a device interface of the computer such that the computer reads the program and executes the methods implemented by the program. The codes may include functional codes associated with a function defining functions necessary to execute the methods or the like, and include control codes associated with an execution procedure necessary for the processor of the computer to execute the functions according to a predetermined procedure. In addition, the codes may further include memory reference codes indicating at which location (address number) of the computer's internal or external memory, additional information or media required for the computer's processor to execute the functions can be referenced. In addition, when the processor of the computer needs to communicate with any other computer or server located remotely to execute the above functions, codes may further include communication-related codes for how to communicate with any other remote computer or server using a communication module of the computer, and what information or media to transmit/receive during communication.

The steps of a method or algorithm described in connection with the embodiments of the present disclosure may be implemented directly in hardware, in a software module executed by hardware, or in a combination thereof. The software module may reside in a random access memory (RAM), a read only memory (ROM), an erasable programmable ROM (EPROM), an electrically erasable programmable ROM (EEPROM), a flash memory, a hard disk, a removable disk, a CD-ROM, or in a computer readable recording medium that is well known in the art.

Unlike the existing eosinophilia diagnosis method that determines only the level of eosinophils in the blood, according to the inventive concept, only the chemical properties of the granules, which are problematic among the chemical properties of eosinophils, are extracted, thereby diagnosing various diseases accompanied by an increase in eosinophils (eosinophilia, chug-Strauss syndrome).

The algorithm of processing Raman data of eosinophils using statistical machine learning enables classification for each component in complex Raman spectral data, enabling comparison for each component between eosinophils and diagnosing eosinophilic asthma.

The extraction of granular data using the algorithm of processing Raman data of eosinophils of the inventive concept can enable accurate comparison for the difference between normal eosinophils and diseased eosinophils, resulting in inference of the composition of changed amino acids and lipids. It is possible to reduce the comparative mean error by including data of background, cell wall and nucleus.

Since the algorithm of processing Raman data of eosinophils according to the inventive concept is an algorithm for extracting granule data of eosinophils, the algorithm is applicable not only to eosinophils but also to other granular cell-related diseases.

However, effects of the inventive concept are may not be limited to the above-described effects. Although not described herein, other effects of the inventive concept can be clearly understood by those skilled in the art from the following description.

While the inventive concept has been described with reference to embodiments, it will be apparent to those skilled in the art that various changes and modifications may be made without departing from the spirit and scope of the inventive concept. Therefore, it should be understood that the above embodiments are not limiting, but illustrative.

What is claimed is:

1. A method for processing Raman data of eosinophil based on artificial intelligence, the method being executed by a device, the method comprising:
generating Raman data by performing Raman analysis using a specific wavelength on eosinophils isolated from blood of a diagnosed person;
pre-processing the generated Raman data;
assigning a weight to the pre-processed Raman data for each of components including a nucleus, a cell membrane, a granule, and a background;
classifying data for each of the components based on a result of assigning the weight;
extracting data in which the component is the granule based on a classified result; and
determining whether a specific disease has occurred in the diagnosed person through eosinophil characteristics of the diagnosed person based on the extracted data.

2. The method of claim 1, wherein the Raman data is data in which two-dimensional data mapped for each point of the specific wavelength is arranged in an order corresponding to a traveling direction of the specific wavelength, and
wherein the two-dimensional data mapped for each point includes different Raman spectra.

3. The method of claim 2, wherein the assigning of the weight includes
converting the two-dimensional data mapped for each point into one-dimensional data by arranging the two-dimensional data in a line;
extracting representative data for each of the components from the converted one-dimensional data; and
assigning a Raman spectrum at a point corresponding to each extracted representative data as a weight for the each extracted representative data.

4. The method of claim 3, wherein the classifying of the data includes performing cluster classification for each of the components using k-means clustering,
wherein the extracting of the representative data includes performing data labeling by giving different labels to the components, determining whether a label labeled in the data is a label corresponding to the granule, and extracting only data labeled with a label corresponding to the granule among the labeled data, based on a result of the determination.

5. The method of claim 4, wherein the extracted data is Raman data in which the component is the granule among the pre-processed Raman data.

6. The method of claim 1, wherein the determining of whether the specific disease has occurred includes analyzing the extracted data using an artificial intelligence-based first model and determining whether eosinophil characteristics of the diagnosed person are within a normal range or an abnormal range to determine whether the specific disease has occurred.

7. The method of claim 6, wherein the first model is built by learning a first Raman data processing process for a plurality of eosinophils isolated from the blood of a plurality of existing patients with the specific disease, a second Raman data processing process for a plurality of eosinophils isolated from the blood of a plurality of normal people without the specific disease, the eosinophil characteristics of the plurality of existing patients acquired by performing the first Raman data processing process and the eosinophil characteristics of the plurality of normal people acquired by performing the second Raman data processing process.

8. The method of claim 1, further comprising:
generating an artificial intelligence-based second model using learning data for a plurality of existing patients; and
predicting a cause of occurrence of the specific disease of a new patient by applying the granule data of the new patient determined as having the specific disease to the second model,
wherein the learning data includes
granule data extracted through Raman analysis on eosinophils isolated from blood of each of the plurality of existing patients as input data, and
data indicating whether or not each of the plurality of existing patients has the specific disease, as output data.

9. The method of claim 8, wherein the predicting of the cause of occurrence of the specific disease includes
classifying an activation level of eosinophilic granule and a type of proteins constituting the eosinophilic granule based on the granule data of the new patient; and
predicting a cause of occurrence of the specific disease for the new patient based on the classified activation level and the type of proteins.

10. The method of claim 8, wherein the input data further includes treatment and treatment result data of each of the plurality of existing patients, and
wherein the second model is trained by performing labeling for each cause of the specific disease using the treatment and treatment result data.

11. The method of claim 10, wherein the predicting of the cause of occurrence of the specific disease includes classifying the cause of the specific disease diagnosed in the new patient into at least one label among a plurality of labels, and
wherein the plurality of labels include congenital, secondary, primary and idiopathic.

12. The method of claim 8, wherein the input data further includes gender and age data of each of the plurality of existing patients, and
wherein the second model has learned symptom levels of the specific disease by gender and age using the gender and age data.

13. The method of claim 12, wherein the predicting of the cause of occurrence of the specific disease includes predicting a likelihood that a symptom of the specific disease diagnosed in the new patient worsens based on gender and age of the new patient.

14. The method of claim 8, wherein the input data further includes granule data of a patient who has been cured of the specific disease or granule data of a normal person without the specific disease.

15. A computer-readable recording medium storing a computer program for executing the method of claim 1 in combination with hardware that is a computer.
